(19) United States
(12) Patent Application Publication
(10) Pub. No.: US 2023/0117066 A1
(43) Pub. Date: Apr. 20, 2023

(54) NEUROSTIMULATOR TRIALING PATIENT ALERT SYSTEM

(71) Applicant: Medtronic, Inc., Minneapolis, MN (US)

(72) Inventors: Michael E Winek, Minneapolis, MN (US); Kunal J Paralikar, Roseville, MN (US); Chika Chima-Nafiu, Fridley, MN (US); Courtney J. Oakland, Golden Valley, MN (US); Lucien Bell N. Solefack, Inver Grove Heights, MN (US); Vincent A. Roczniak, Maple Grove, MN (US); Christina Leung, Minneapolis, MN (US); Troy A. Thielen, Shoreview, MN (US)

(21) Appl. No.: 17/933,395

(22) Filed: Sep. 19, 2022

Related U.S. Application Data
(60) Provisional application No. 63/255,671, filed on Oct. 14, 2021.

(51) Int. Cl. A61N 1/08; A61B 5/11; A61B 5/00

(52) U.S. Cl. CPC A61N 1/08; A61B 5/1118; A61B 5/1123; A61B 5/746; A61N 2001/083

(57) ABSTRACT

In some examples, a medical system includes one or more trialing leads implanted within a patient, one or more sensors configured to determine a value for a sensed parameter indicative of an activity level of the patient, and processing circuitry. The processing circuitry may be configured to receive the value from the one or more sensors, determine whether the value is outside a threshold range, and—in response to determining that the value is outside the threshold range—generate information indicating a status of the one or more trialing leads. In some examples, processing circuitry may be configured to output an alert warning that patient movement could dislodge, or has already dislodged, the one or more trialing leads.

This application claims the benefit of U.S. Provisional Application Ser. No. 63/255,671, filed Oct. 14, 2021, the entire content of which is incorporated herein by reference.

### TECHNICAL FIELD

This disclosure generally relates to electrical stimulation therapy, and more specifically, control of electrical stimulation therapy.

### BACKGROUND

Medical devices may be external or implanted and may be used to deliver electrical stimulation therapy to patients via various tissue sites to treat a variety of symptoms or conditions such as chronic pain, tremor, Parkinson's disease, epilepsy, urinary or fecal incontinence, sexual dysfunction, obesity, or gastroparesis. A medical device may deliver electrical stimulation therapy via one or more leads that include electrodes located proximate to target locations associated with the brain, the spinal cord, pelvic nerves, peripheral nerves, or the gastrointestinal tract of a patient. Stimulation proximate the spinal cord, proximate the sacral nerve, within the brain, and proximate peripheral nerves are often referred to as spinal cord stimulation (SCS), sacral neuromodulation (SNM), deep brain stimulation (DBS), and peripheral nerve stimulation (PNS), respectively.

In some cases, before a medical device to provide electrical stimulation therapy is permanently installed in vivo, a patient undergoes a trialing period where the medical device is worn outside the body, with only the leads extending inside the body.

### SUMMARY

Systems, devices, and techniques are described for generating information indicating a status of one or more trialing leads, such as when a patient is undergoing an activity. As an example, the techniques may include alerting a patient undergoing neurostimulation trialing of movements which may cause the one or more trialing leads to move in a way that jeopardizes the effectiveness of the trialing. During trialing of neurostimulators, patients tend to move around. This causes trialing leads to become dislodged, disconnected, or to cause discomfort to the patient. Dislodged and disconnected trialing leads may no longer provide adequate therapy to a patient or may provide incomplete information from the trial.

In one or more examples described in this disclosure, processing circuitry may determine whether activity of the patient may be such that the one or more leads may dislodge or such that there is low confidence in the accuracy of signals sensed by the one or more leads. A patient may utilize such information to adjust or change activity. In this way, rather than modifying leads or using additional ways to secure the trialing leads, the example techniques generate information that allow the patient to take proactive action to minimize chances of dislodging the leads and potentially increase accuracy of signals sensed by the trialing leads.

Although the examples are described with respect to trialing leads, the example techniques may be applicable more generally to medical systems. For instance, the techniques may be applicable to components of medical systems that are temporally or chronically implanted in a patient.

In some examples, a medical system includes one or more trialing leads implanted within a patient, one or more motion sensors configured to measure a value for a sensed parameter indicative of an activity level of the patient, and processing circuitry configured to: receive the value from the one or more motion sensors; determine whether the value is outside a threshold range; in response to determining that the value is outside the threshold range, generate information indicating a status of the one or more trialing leads.

In some examples, a method comprises: receiving from one or more motion sensors, by processing circuitry, a value for a sensed parameter indicative of an activity level of a patient; determining, by processing circuitry, that the value is outside a threshold range; and responsive to determining that the value is outside the threshold range, generating, by processing circuitry, information indicating a status of one or more trialing leads implanted within the patient.

In some examples, a computer-readable storage medium stores instructions thereon that when executed cause one or more processors to: receive from one or more sensors a value for a sensed parameter indicative of an activity level of a patient; determine that the value is outside a threshold range; and responsive to determining that the value is outside the threshold range, generate information indicating a status of one or more trialing leads implanted within the patient.

In some examples, a device includes means for receiving from one or more sensors a value for a sensed parameter indicative of an activity level of a patient; means for determining that the value is outside a threshold range; and means for generating information indicating a status of one or more trialing leads implanted within the patient responsive to determining that the value is outside the threshold range.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram illustrating an example system that includes a medical device configured to generate information indicating a status of one or more trialing leads according to the techniques of the disclosure.

FIG. 2 is a block diagram of the example the medical device of FIG. 1.

FIG. 3 is a conceptual diagram illustrating an example alert on a user interface to warn a user of excessive activity levels.

FIG. 4 is a flowchart illustrating an example technique for preventing dislodged trialing leads according to the techniques of this disclosure.

### DETAILED DESCRIPTION

The disclosure describes devices, systems, and techniques for preventing leads of a medical device, such as a neurostimulator, from being dislodged during trialing periods.

When in normal use, a medical device such as a neurostimulator, may be positioned entirely within a patient, and leads delivering therapy to the patient may be secured within the body. When secured within the body, a medical device and any leads connected to the medical device may be sheltered from outside influence like bumping against solid objects, catching on hooked objects, or otherwise being interfered with. Furthermore, a patient's own activity level will have less effect on the integrity of the medical device and connected leads while secured inside the body.

However, many patients undergo a trialing period before receiving the surgery necessary to implant the medical device to see if the therapy the medical device can provide will be useful. During the trialing period, i.e., for a neurostimulator, the main body of the neurostimulator may remain outside the body, while the trialing leads may enter the body at an entry site to deliver therapy. The neurostimulator may be worn on a belt, in a pocket, be secured to the skin, or otherwise be positioned near the entry site. Leads may comprise long wires, and may be connected to the medical device (e.g., neurostimulator), pierce through the patient's skin, and enter the patient to deliver therapy. The long wires of the leads may be prone to catching on objects or being twisted out of position by the patient's movements.

Great time and expense may be spent attempting to secure the leads in place on the patient's skin, or providing more flexible and durable leads to avoid breakage altogether. However, securing the leads to the patient's skin may aggravate the patient's skin in that area. Furthermore, it may be undesirable to make leads so strong that, if caught on an external object, the patient's tissues to which the leads are connected tear inside or outside the body before the leads tear or fail. In addition, as a trialing period is meant to be temporary, the connections between the medical device, leads, and patient tissues may not be so strong as to make them difficult to separate. Therefore, excess activity from the patient themself is always at risk of compromising the positioning of the leads, causing discomfort, and compromising the effectiveness of the therapy during the trialing period. It may be most advantageous to change the behavior of the patient through warnings and alerts before their behavior dislodges the neurostimulators and causes discomfort.

In some examples, this disclosure describes devices, systems, and techniques for monitoring patient parameters indicative of one or more motions of a patient indicating an activity level of the patient. The activity level may be compared to predetermined threshold ranges to determine a status of the trialing leads. In some examples, the monitoring of the patient parameters may be by using one or more motion sensors.

The medical device may be configured to output an alert warning that patient movement could dislodge—or has dislodged—the trialing leads. The medical device may have motion sensors incorporated into the medical device's circuitry, or the medical device may make use of motion sensors on another device with which the medical device is in communication. The motion sensors may be in communication with circuitry configured to send messages to an application on a patient's phone, and circuitry configured to save and report data on patient motion during neurostimulator device use.

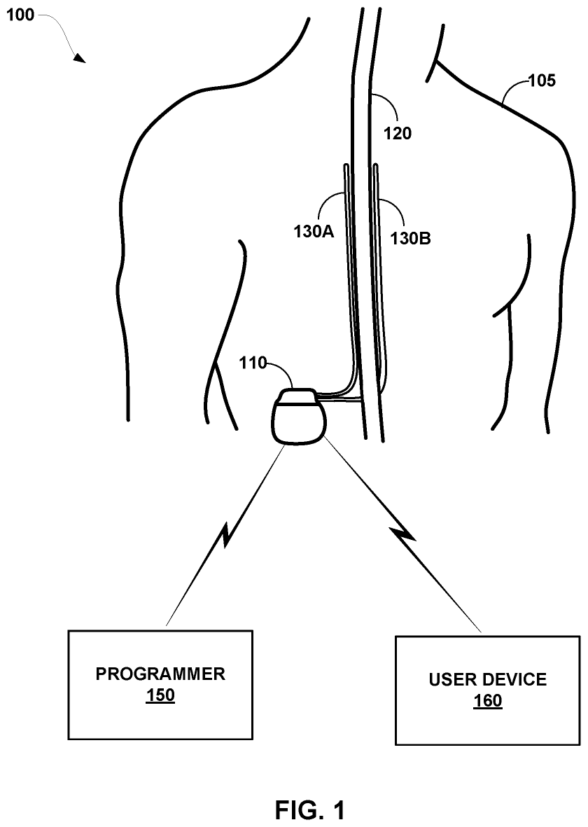

FIG. 1 is a conceptual diagram illustrating an example system that includes a medical device 110 configured to generate information indicating a status of one or more trialing leads 130A and/or 130B (collectively, “leads 130”) implanted within a patient, according to the techniques of the disclosure. Medical device 110 generating information indicating a status of one or more trialing leads is provided for example purposes only, and should not be considered limiting. In general, processing circuitry may be configured to generate information indicating a status of one or more trialing leads, and the processing circuitry may be part of medical device 110, part of another device, or may be partially located in different devices. For example, processing circuitry may be a part of medical device 110, programmer 150, user device 160, or any combination thereof.

In the example shown in FIG. 1, medical device 110 is configured to deliver spinal cord stimulation (SCS) therapy. Although the techniques described in this disclosure are generally applicable to a variety of medical devices including external and medical devices, application of such techniques to medical devices and, more particularly, implantable electrical stimulators (e.g., neurostimulators) will be described for purposes of illustration. More particularly, the disclosure will refer to an implantable spinal cord stimulation (SCS) system for purposes of illustration, but without limitation as to other types of medical devices or other therapeutic applications of medical devices.

A medical device may deliver electrical stimulation to a patient where the electrical stimulation includes a pulse train with control pulses configured to elicit sensed electrical signals as well as informed pulses configured to provide therapy. One type of electrical signal elicited by the control pulses may be evoked compound action potentials (ECAPs). For ease of description, examples of the disclosure are primarily described with regard to the elicited signals being ECAPs although other types of sensed elicited electrical signals such as evoked compound muscle action potentials (eCMAPs) or local field potentials (LFPs) are contemplated. The use of ECAPs, eCMAPs, or LFPs should not be considered limiting.

Although electrical stimulation is generally described herein in the form of electrical stimulation pulses, electrical stimulation may be delivered in non-pulse form in other examples. For example, electrical stimulation may be delivered as a signal having various waveform shapes, frequencies, and amplitudes. Therefore, electrical stimulation in the form of a non-pulse signal may be a continuous signal than may have a sinusoidal waveform or other continuous waveform.

As shown in FIG. 1, system 100 includes a medical device 110 in communication with a user device 160, leads 130, and programmer 150 shown in conjunction with a patient 105, who is ordinarily a human patient. In the example of FIG. 1, medical device 110 is an electrical stimulator that is configured to generate and deliver electrical stimulation therapy to patient 105 via one or more electrodes of leads 130, e.g., for relief of chronic pain or other symptoms. In other examples, medical device 110 may be coupled to a single lead carrying multiple electrodes or more than two leads each carrying multiple electrodes. Medical device 110 may be configured to generate and deliver control pulses configured to elicit ECAP signals that may or may not contribute to the therapy of informed pulses. The control pulses may be non-therapeutic in some examples.

Medical device 110 may be configured to sense electrical signals elicited by the delivered control pulses via one or more electrodes on leads 130 and deliver a train of informed pulses to provide therapy to a patient. Medical device 110 may be a temporary, or trial, stimulator used to screen or evaluate the efficacy of electrical stimulation for chronic therapy. In one example, medical device 110 is an external device coupled to percutaneously implanted leads.

Electrical stimulation energy, which may be constant current or constant voltage-based pulses, for example, is delivered from medical device 110 to one or more target tissue sites of patient 105 via one or more electrodes (not shown) of implantable leads 130. In the example of FIG. 1, leads 130 carry electrodes that are placed adjacent to the target tissue of spinal cord 120. One or more of the electrodes may be disposed at a distal tip of a lead 130 and/or at other positions at intermediate points along the lead. Leads 130 may be implanted and coupled to medical device 110. The electrodes may transfer electrical stimulation generated by an electrical stimulation generator in medical device 110 to tissue of patient 105. Although leads 130 may each be a single lead, lead 130 may include a lead extension or other segments that may aid in implantation or positioning of lead 130.

The electrodes of leads 130 may be electrode pads on a paddle lead, circular (e.g., ring) electrodes surrounding the body of the lead, conformable electrodes, segmented electrodes (e.g., electrodes disposed at different circumferential positions around the lead instead of a continuous ring electrode), any combination thereof (e.g., ring electrodes and segmented electrodes) or any other type of electrodes capable of forming unipolar, bipolar or multipolar electrode combinations for therapy. Ring electrodes arranged at different axial positions at the distal ends of lead 130 will be described for purposes of illustration.

The deployment of electrodes via leads 130 is described for purposes of illustration, but arrays of electrodes may be deployed in different ways. For example, a housing associated with a leadless stimulator may carry arrays of electrodes, e.g., rows and/or columns (or other patterns), to which shifting operations may be applied. Such electrodes may be arranged as surface electrodes, ring electrodes, or protrusions. As a further alternative, electrode arrays may be formed by rows and/or columns of electrodes on one or more paddle leads. In some examples, electrode arrays may include electrode segments, which may be arranged at respective positions around a periphery of a lead, e.g., arranged in the form of one or more segmented rings around a circumference of a cylindrical lead. In other examples, one or more of leads 130 are linear leads having 8 ring electrodes along the axial length of the lead. In another example, the electrodes are segmented rings arranged in a linear fashion along the axial length of the lead and at the periphery of the lead.

The stimulation parameter of a therapy stimulation program that defines the stimulation pulses of electrical stimulation therapy by medical device 110 through the electrodes of leads 130 may include information identifying which electrodes have been selected for delivery of stimulation according to a stimulation program, the polarities of the selected electrodes, i.e., the electrode combination for the program, and voltage or current amplitude, pulse frequency, pulse width, pulse shape of stimulation delivered by the electrodes. These stimulation parameters of informed pulses are typically predetermined parameter values determined prior to delivery of the informed pulses. However, in some examples, system 100 may change one or more parameter values automatically based on one or more factors or based on user input.

The stimulation signals (e.g., one or more stimulation pulses or a continuous stimulation waveform) defined by the parameters of each ECAP test stimulation program are configured to evoke a compound action potential from nerves. In some examples, the ECAP test stimulation program may define when the non-informing and informing control pulses are to be delivered to the patient based on the frequency and/or pulse width of the informed pulses. However, the stimulation defined by each ECAP test stimulation program may or may not be intended to provide or contribute to therapy for the patient. In an example where the control pulses contribute to or provide therapy for the patient, the ECAP test stimulation program may also be used in place of, or be the same as, a therapy stimulation program.

Although FIG. 1 is directed to SCS therapy, e.g., used to treat pain, in other examples system 100 may be configured to treat any other condition that may benefit from electrical stimulation therapy. For example, system 100 may be used to treat tremor, Parkinson's disease, epilepsy, a pelvic floor disorder (e.g., urinary incontinence or other bladder dysfunction, fecal incontinence, pelvic pain, bowel dysfunction, or sexual dysfunction), obesity, gastroparesis, auto-immune conditions (e.g., diabetes), rheumatoid arthritis, or psychiatric disorders (e.g., depression, mania, obsessive compulsive disorder, anxiety disorders, and the like). In this manner, system 100 may be configured to provide therapy taking the form of deep brain stimulation (DBS), peripheral nerve stimulation (PNS), peripheral nerve field stimulation (PNFS), cortical stimulation (CS), pelvic floor stimulation, gastrointestinal stimulation, or any other stimulation therapy capable of treating a condition of patient 105.

Medical device 110 is configured to deliver electrical stimulation therapy to patient 105 via selected combinations of electrodes carried by one or both of leads 130, alone or in combination with an electrode carried by or defined by an outer housing of medical device 110. The target tissue for the electrical stimulation therapy may be any tissue affected by electrical stimulation, which may be in the form of electrical stimulation pulses or continuous waveforms. In some examples, the target tissue includes nerves, smooth muscle or skeletal muscle. In the example illustrated by FIG. 1, the target tissue is tissue proximate spinal cord 120, such as within an intrathecal space or epidural space of spinal cord 120, or, in some examples, adjacent nerves that branch off spinal cord 120. Leads 130 may be introduced into spinal cord 120 in via any suitable region, such as the thoracic, cervical or lumbar regions. Stimulation of spinal cord 120 may, for example, prevent pain signals from traveling through spinal cord 120 and to the brain of patient 105. Patient 105 may perceive the interruption of pain signals as a reduction in pain and, therefore, efficacious therapy results. In other examples, stimulation of spinal cord 120 may produce paresthesia which may be reduce the perception of pain by patient 105, and thus, provide efficacious therapy results.

A user, such as a clinician or patient 105, may interact with a user interface of a programmer 150 to program medical device 110. Programming of medical device 110 may refer generally to the generation and transfer of commands, programs, or other information to control the operation of medical device 110. In this manner, medical device 110 may receive the transferred commands and programs from programmer 150 to control electrical stimulation therapy and control stimulation (e.g., control pulses). For example, programmer 150 may transmit therapy stimulation programs, ECAP test stimulation programs, stimulation parameter adjustments, therapy stimulation program selections, ECAP test program selections, user input, or other information to control the operation of medical device 110, e.g., by wireless telemetry or wired connection. In some examples, the therapy stimulation adjustments may be made in response to a detected activity level of the patient, or in response to a changed status of the trialing leads.

In some cases, programmer 150 may be characterized as a physician or clinician programmer if it is primarily intended for use by a physician or clinician. In other cases, programmer 150 may be characterized as a patient programmer if it is primarily intended for use by a patient. A patient programmer may be generally accessible to patient 105 and, in many cases, may be a portable device that may accompany patient 105 throughout the patient's daily routine. For example, a patient programmer may receive input from patient 105 when the patient wishes to terminate or change electrical stimulation therapy. In general, a physician or clinician programmer may support selection and generation of programs by a clinician for use by medical device 110, whereas a patient programmer may support adjustment and selection of such programs by a patient during ordinary use. In other examples, programmer 150 may include, or be part of, an external charging device that recharges a power source of medical device 110. In this manner, a user may program and charge medical device 110 using one device, or multiple devices. Although programmer 150 and user device 160 are illustrated as separate components, in some examples, programmer 150 may be a patient programmer that doubles as user device 160.

User device 160 may be any suitable communication or computing device, such as a mobile phone, wearable, and/or non-wearable computing device capable of communicating with medical device 110. For example, user device 160 may include a conventional mobile phone, a smart phone, a tablet computer, a computerized watch, a computerized glove or gloves, a personal digital assistant, a virtual assistant, a gaming system, a media player, an e-book reader, a television or television platform, an exercise machine interface, or navigation, information and/or entertainment system for a bicycle, automobile or other vehicle, a laptop or notebook computer, or any other type of computing device that may perform operations in accordance with one or more aspects of the present disclosure. User device 160 may support communication services over packet-switched networks, e.g., the public Internet. User device 160 may also support communication services over circuit-switched networks, e.g., the public switched telephone network (PSTN). User device 160 may use network interfaces (such as optical transceivers, radio frequency (RF) transceivers, Wi-Fi or Bluetooth radios, or the like), telephony interfaces, or any other type of device that can send and receive information to wirelessly communicate with external systems, e.g., medical device 110, programmer 150. Each of user device 160 may be operated by a user that may be a patient undergoing trialing for a medical device 110. User device 160 may include one or more sensors configured to determine a value for a sensed parameter indicative of an activity level of the patient.

Information may be transmitted between programmer 150 and medical device 110. Therefore, medical device 110 and programmer 150 may communicate via wireless communication using any techniques known in the art. Examples of communication techniques may include, for example, radiofrequency (RF) telemetry and inductive coupling, but other techniques are also contemplated. In some examples, programmer 150 may include a communication head that may be placed proximate to the patient's body near the medical device 110 implant site to improve the quality or security of communication between medical device 110 and programmer 150. Communication between programmer 150 and medical device 110 may occur during power transmission or separate from power transmission.

In some examples, medical device 110, in response to commands from programmer 150, delivers electrical stimulation therapy according to a plurality of therapy stimulation programs to a target tissue site of the spinal cord 120 of patient 105 via electrodes (not depicted) on leads 130. In some examples, medical device 110 may modify therapy stimulation programs as therapy needs of patient 105 evolve over time. For example, the modification of the therapy stimulation programs may compensate for movement of the leads. When patient 105 receives the same therapy for an extended period, the efficacy of the therapy may be reduced. In some cases, parameters of the informed pulses may be automatically updated.

Efficacy of electrical stimulation therapy may be indicated by one or more characteristics (e.g., an amplitude of or between one or more peaks or an area under the curve of one or more peaks) of an action potential that is evoked by a stimulation pulse delivered by medical device 110 (i.e., a characteristic of the ECAP signal). Electrical stimulation therapy delivery by leads 130 of medical device 110 may cause neurons within the target tissue to evoke a compound action potential that travels up and down the target tissue, eventually arriving at sensing electrodes of medical device 110. Furthermore, control stimulation may also elicit at least one ECAP, and ECAPs responsive to control stimulation may also be a surrogate for the effectiveness of the therapy. The amount of action potentials (e.g., number of neurons propagating action potential signals) that are evoked may be based on the various parameters of electrical stimulation pulses such as amplitude, pulse width, frequency, pulse shape (e.g., slew rate at the beginning and/or end of the pulse), etc. The slew rate may define the rate of change of the voltage and/or current amplitude of the pulse at the beginning and/or end of each pulse or each phase within the pulse. For example, a very high slew rate indicates a steep or even near vertical edge of the pulse, and a low slew rate indicates a longer ramp up (or ramp down) in the amplitude of the pulse. In some examples, these parameters may contribute to an intensity of the electrical stimulation. In addition, a characteristic of the ECAP signal (e.g., an amplitude) may change based on the distance between the stimulation electrodes and the nerves subject to the electrical field produced by the delivered control pulses.

In the example of FIG. 1, medical device 110 is described as performing a plurality of processing and computing functions. However, programmer 150 or user device 160 instead may perform one, several, or all of these functions. In this alternative example, medical device 110 functions to relay sensed signals to programmer 150 for analysis. For example, medical device 110 may relay a value for a sensed parameter indicative of a patient activity level to programmer 150. Programmer 150 may determine whether the value is outside a predetermined range of values for the sensed parameter, and in response to the determination, programmer 150 may generate information indicating a status of the one or more trialing leads 130.

In some examples, programmer 150, lead 130, user device 160, and/or medical device 110 may include one or more sensors configured to allow medical device 110 to monitor one or more parameters of patient 105, such as patient activity, pressure, temperature, or other characteristics. Additionally, or alternatively, the one or more sensors may be separate from that of lead 130 (e.g., as a separate sensor communicatively coupled to medical device 110). The one or more sensors may be provided in addition to therapy delivery by lead 130. In some examples, medical device 110 may generate information indicating a status of the leads 130 based on one or more parameters monitored by the one or more sensors. In some examples, the one or more sensors may be configured to determine a value of a sensed parameter indicative of an activity level of a patient. In some examples, medical device 110 may output an alert warning that patient movement could dislodge leads 130 or otherwise compromise the effectiveness of the therapy. Medical device 110, a server, or another storage device may include a buffer or other memory structure which temporarily or permanently stores sensor signals and/or parameter values of patient 105.

The one or more sensors may be configured to measure a value for a sensed parameter indicative of an activity level of patient 105. The one or more sensors may include one or more accelerometers, strain gauges, microelectromechanical systems (MEMS) gyroscopes, thermometers, heart rate monitors, electrodes, or other sensors configured to measure a value for a sensed parameter of patient 105.

In some examples, the one or more sensors configured to monitor one or more parameters of patient 105 include one or more motion sensors including one or more accelerometers configured to measure an acceleration of patient 105 indicative of a motion of patient 105. In some examples, the accelerometer may collect a three-axis accelerometer signal indicative of patient 105's movements within a three-dimensional Cartesian space. For example, the accelerometer signal may include a vertical axis accelerometer signal vector, a lateral axis accelerometer signal vector, and a frontal axis accelerometer signal vector. The vertical axis accelerometer signal vector may represent an acceleration of patient 105 along a vertical axis, the lateral axis accelerometer signal vector may represent an acceleration of patient 105 along a lateral axis, and the frontal axis accelerometer signal vector may represent an acceleration of patient 105 along a frontal axis. In some cases, the vertical axis substantially extends along a torso of patient 105 from a neck of patient 105 to a waist of patient 105, the lateral axis extends across a chest of patient 105 perpendicular to the vertical axis, and the frontal axis extends outward from and through the chest of patient 105, the frontal axis being perpendicular to the vertical axis and the lateral axis.

The three-axis accelerometer signal may be indicative of patient 105's activity level. For example, higher accelerometer signal values may indicate a higher activity level, and lower accelerometer signal values may indicate a lower activity level.

Processing circuitry of medical device 110 may use the three-axis accelerometer signal to differentiate different types of activities. In some examples, a vertical axis accelerometer signal may alternate from positive to negative repeatedly over a period of time, indicating that patient 105 may be jumping. In some examples, a lateral axis accelerometer signal may alternate from positive to negative repeatedly over a period of time, indicating that patient 105 may be dancing. In some examples, a frontal axis accelerometer signal may experience a sudden spike or valley, indicating that patient 105 has sped up or stopped suddenly.

In some examples, the one or more sensors configured to allow medical device 110 to monitor one or more parameters of patient 105 include one or more motion sensors including one or more strain gauges configured to measure a strain of one or more parts of patient 105. In some examples, one or more strain gauges may collect a one or more strain signals indicative of patient 105's movements and stretching. For example, the one or more strain gauges may include strain gauges oriented along a vertical axis, a lateral axis, a frontal axis, and/or at an angle to any one or more of the aforementioned axes, to collect a vertical strain signal, a lateral strain signal, a frontal strain signal, and/or any other strain signal to detect strain of patient 105. The vertical, lateral, and frontal axes may be identical to the ones described above for accelerometers.

The one or more strain signals may be indicative of patient 105's activity level. For example, a detected strain signal may indicate that patient 105 is stretching their body in a certain direction or manner. Higher measured strain may indicate a higher activity level, and lower measured strain may indicate a lower activity level.

Processing circuitry of medical device 110 may use the one or more strain signals to differentiate different types of stretches. In some examples, where the one or more strain gauges are positioned on the back of patient 105, a vertical strain value may increase, while a lateral strain value may not change, indicating that the patient may be bending forward. In some examples, where one or more strain gauges are positioned on the back of patient 105, a lateral strain value may increase, while a vertical strain value may not change, indicating that the patient may be twisting their upper body. In some examples where one or more strain gauges are positioned on the left side of patient 105, a vertical strain value may increase, while a frontal strain value may not change, indicating that patient 105 may be bending over their right side and reaching their left arm over their head. In a similar way, the one or more strain gauges may be configured to detect any type of stretching of patient 105.

In some examples, the one or more sensors configured to allow medical device 110 to monitor one or more parameters of patient 105 include one or more gyroscopes configured to measure the extent and rate of patient 105's rotation in space (i.e., roll, pitch and yaw). In some examples, the one or more gyroscopes may collect one or more gyroscope signals indicative of patient 105's movements in cartesian space. The one or more gyroscopes may work in tandem with the one or more accelerometers to provide a more complete, six-axis acceleration sensor for patient 105. The one or more gyroscope signals may be indicative of patient 105's activity level, where higher gyroscope signal values may indicate a higher activity level and lower gyroscope signal values may indicate a lower activity level. Processing circuitry of medical device 110 may use the one or more gyroscope signals to determine roll, pitch, yaw, and/or angular acceleration for patient 105.

In some examples, the one or more sensors configured to allow medical device 110 to monitor one or more parameters of patient 105 include one or more thermometers configured to measure a temperature of patient 105. Higher temperatures may indicate a higher activity level, and lower temperatures may indicate a lower activity level. Processing circuitry of medical device 110 may use temperature data in conjunction with other sensor data when determining an activity type or level for patient 105. For example, a high accelerometer signal and a high temperature may be a better indication of an activity level of a patient than a high accelerometer signal and a normal temperature.

In some examples, the one or more sensors configured to monitor one or more parameters of patient 105 include one or more heart rate monitors configured to a measure a heart rate of patient 105. A higher heart rate may indicate a higher activity level, and a lower heart rate may indicate a lower activity level. Processing circuitry of medical device 110 may use heart rate data in conjunction with other sensor data when determining an activity type or level for patient 105. For example, a high accelerometer signal and a high heart rate may be a better indication of an activity level of a patient than a high accelerometer signal and a normal heart rate.

Medical device 110 may include processing circuitry configured to receive the value of the sensed parameter and determine whether that value is outside a threshold range. Processing circuitry of medical device 110 may track the value of the sensed parameter over a period of time (e.g., hours, days, weeks), to determine if the value is outside the threshold range at any point during the period of time. Medical device 110 may record instances when the value is outside the threshold range in a database in memory, including the sensed parameter, the parameter value, and a time stamp. In some examples, medical device 110 may record all sensor values in a database in memory over a period of time to create profile data for patient 105. The profile data for patient 105 may be compared to profile data for a plurality of patients to create average and threshold activity levels for patients. In some examples, threshold ranges for activity levels may be determined by a physician specifically for the patient on which medical device 110 is installed. Threshold ranges may be calibrated for individual patients based on the positioning of medical device 110 after installation. In some examples, the threshold ranges are pre-set values based on development testing of medical device 110. The preset values for threshold ranges may be based on average values for threshold ranges across a wide variety of patients. In some examples, the threshold ranges are regularly updated based on profile data from a plurality of patients using medical device 110.

Processing circuitry of medical device 110 may determine that a value of a sensed parameter is outside a threshold range, and in response, generate information indicating a status of the trialing leads. In some examples, information indicating a status of the trialing leads may be generated using ECAP signal data. ECAP signals may be sensed by electrodes of medical device 110 in response to control stimulation pulses delivered by medical device 110. Medical device 110 may store ECAP signals in a buffer and an algorithm executed on processing circuitry of medical device 110 may analyze the signals. The algorithm may detect a sudden change in the amplitude of ECAPs in the ECAP signal, indicating a sudden change in position of the trialing leads. In some examples, processing circuitry of medical device 110 may generate information indicating that there is a low confidence in an accuracy of a signal sensed by the trialing leads (e.g., accuracy of a signal sensed by the trialing leads). For example, an amplitude of a measured ECAP signal may drop so far, that the signal is more likely to be noise than actual ECAPs. In this case, the amplitude has dropped so far that ECAPs are effectively no longer being detected. In this case, the algorithm may determine that the trialing leads have become completely dislodged and can no longer provide effective therapy.

Using ECAPs is one example way to determine status of the trialing leads. In some examples, information indicating a status of the trialing leads may be generated using impedance data for medical device 110. Medical device 110 and/or processing circuitry thereof may be configured to detect an impedance of the trialing leads. A sudden change in the detected impedance of the trialing leads may be indicative of a movement of the trialing leads within patient 105. In some examples, information indicating a status of the trialing leads may be generated using resistance data for medical device 110. Processing circuitry of medical device 110 may be configured to detect a resistance of the trialing leads. A sudden change in the detected resistance of the trialing leads may be indicative of a movement of the trialing leads within patient 105. In some examples, processing circuitry of medical device 100 may be configured to measure the current through or voltage across the trialing leads or electrodes on the trialing leads. A sudden change in the measured voltage across or current through the trialing leads or electrodes may be indicative of a movement of the trialing leads within patient 105.

In some examples a plurality of electrodes may be disposed on the trialing leads, each configured to deliver therapy to patient 105, and each configured to sense parameters of patient 105. When a change in sensed patient parameter signal is observed across all electrodes simultaneously, it may be an indication of the trialing leads being moved or dislodged. For example, each electrode of the plurality of electrodes may sense an ECAP signal. If the sensed ECAP signal suddenly drops across all electrodes of the plurality of electrodes, it may be an indication that the trialing leads have been moved or dislodged.

Information indicating a status of the trialing leads may be analyzed in conjunction with one or more sensed parameters from one or more sensors to determine an activity level of patient 105 and response thereto. For example, a gyroscope and/or accelerometer may register a sudden twisting motion and at the same time a strain gauge may indicate that patient 105 is stretching. In this example, each electrode of a plurality of electrodes on a trialing lead may simultaneously register a sudden drop in ECAP signal. In response, processing circuitry of medical device 110 may determine that the activity of patient 105 caused the trailing leads to become dislodged.

In some examples, processing circuitry of medical device 110 may be configured to output an alert warning that patient 105 movement could dislodge the trialing leads. For example, processing circuitry of medical device 110 may receive a sensed parameter value that is outside a first threshold range, indicating that the activity level of the patient risks moving or dislodging the trialing leads. Processing circuitry may output an alert to patient 105 via user device 160 or programmer 150, warning patient 105 of the increased risk of moving or dislodging the trialing leads. The patient may then adjust their behavior to avoid moving or dislodging the leads. In some examples, processing circuitry may receive a sensed parameter value that is outside a second threshold range, indicating that the patient has likely dislodged the trialing leads and may need to return to the physician for re-insertion. Processing circuitry may output an alert to patient 105 indicting that the trialing leads have likely been dislodged.

By warning patient 105 of excess movement that may cause trialing leads to become dislodged, the present disclosure allows patient 205 to adjust their behavior, and avoid dislodging or moving the trialing leads, thereby avoiding poor therapy from medical device 110, and also avoiding potential re-insertion of the trialing leads and further discomfort for patient 105.

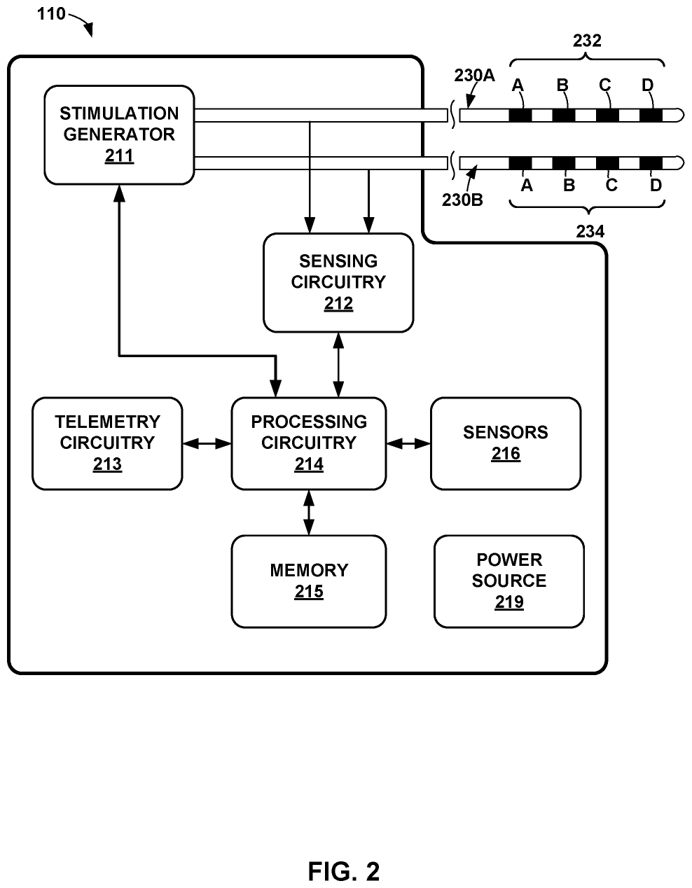

FIG. 2 is a block diagram of the example medical device 110 of FIG. 1. In the example shown in FIG. 2, medical device 110 includes processing circuitry 214, memory 215, stimulation generator 211, sensing circuitry 212, telemetry circuitry 213, sensors 216, and power source 219. Each of these circuits may be or include programmable or fixed function circuitry configured to perform the functions attributed to respective circuitry. For example, processing circuitry 214 may include fixed-function or programmable circuitry, stimulation generator 211 may include circuitry configured to generate stimulation signals such as pulses or continuous waveforms on one or more channels, sensing circuitry 212 may include sensing circuitry for sensing signals, and telemetry circuitry 213 may include telemetry circuitry for transmission and reception of signals. Memory 215 may store computer-readable instructions that, when executed by processing circuitry 214, cause medical device 110 to perform various functions. Memory 215 may be a storage device or other non-transitory medium. Medical device 110 may also contain a switch for coupling sensing circuitry 212 to selected electrodes 232, 234.

In some examples, memory 215 includes one or both of a short-term memory or a long-term memory. Memory 215 may include, for example, random access memories (RAM), dynamic random-access memories (DRAM), static random-access memories (SRAM), magnetic discs, optical discs, flash memories, or forms of electrically programmable memories (EPROM) or electrically erasable and programmable memories (EEPROM). In some examples, memory 215 is used to store program instructions for execution by processing circuitry 14.

Processing circuitry 214 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 214 herein may be embodied as firmware, hardware, software, or any combination thereof. Processing circuitry 214 controls stimulation generator 211 to generate stimulation signals according to therapy stimulation programs and ECAP test stimulation programs stored in memory 215 to apply stimulation parameter values specified by one or more of programs. Although processing circuitry 214 is depicted as within medical device 110, and described below with respect to medical device 110, in some examples, parts or all of processing circuitry may lie on user device 160 or programmer 150.

In the example shown in FIG. 2A, the set of electrodes 232 includes electrodes 232A, 232B, 232C, and 232D, and the set of electrodes 234 includes electrodes 234A, 234B, 234C, and 234D. In other examples, a single lead may include all eight electrodes 232 and 234 along a single axial length of the lead. Processing circuitry 214 also controls stimulation generator 211 to generate and apply the stimulation signals to selected combinations of electrodes 232, 234. In some examples, stimulation generator 211 includes a switch circuit that may couple stimulation signals to selected conductors within leads 230, which, in turn, deliver the stimulation signals across selected electrodes 232, 234. Such a switch circuit may be a switch array, switch matrix, multiplexer, or any other type of switching circuit configured to selectively couple stimulation energy to selected electrodes 232, 234 and to selectively sense bioelectrical neural signals of a spinal cord 120 of the patient (not shown in FIG. 2A) with selected electrodes 232, 234.

In other examples, however, stimulation generator 211 does not include a switch circuit. In these examples, stimulation generator 211 comprises a plurality of pairs of voltage sources, current sources, voltage sinks, or current sinks connected to each of electrodes 232, 234 such that each pair of electrodes has a unique signal circuit. In other words, in these examples, each of electrodes 232, 234 is independently controlled via its own signal circuit (e.g., via a combination of a regulated voltage source and sink or regulated current source and sink), as opposed to switching signals between electrodes 232, 234.

For example, to activate electrode 232A as a cathode, a current source connected to electrode 232A may be turned on and specified with an amount of electric current amplitude to be delivered with each pulse. To activate electrode 232B as an anode, a current sink connected to electrode 232B may be turned on, which causes electrode 232B to sink the amount of current sourced by electrode 232A. Stimulation generator 211 may time-mux different electrode combinations by turning on different sources and sinks, connected to other selected electrodes, at different times. Stimulation generator 211 may also form multi-electrode combinations of multiple cathodes and/or multiple anodes (or one cathode and multiple anodes or one anode and multiple cathodes) by selectively turning on particular sources and sinks, with the total amount of current sourced by the current sources of the cathodes being equal to the total amount of current sunk by the current sinks of the anode. In some examples, the housing of medical device 110 may form an anode.

Electrodes 232, 234 on respective leads 230 may be constructed of a variety of different designs. For example, one or both of leads 230 may include one or more electrodes at each longitudinal location along the length of the lead, such as one electrode at different perimeter locations around the perimeter of the lead at each of the locations A, B, C, and D. In one example, the electrodes may be electrically coupled to stimulation generator 211, e.g., via switch circuitry 210 and/or switching circuitry of the stimulation generator 211, via respective wires that are straight or coiled within the housing of the lead and run to a connector at the proximal end of the lead. In another example, each of the electrodes of the lead may be electrodes deposited on a thin film. The thin film may include an electrically conductive trace for each electrode that runs the length of the thin film to a proximal end connector. The thin film may then be wrapped (e.g., a helical wrap) around an internal member to form the lead 230. These and other constructions may be used to create a lead with a complex electrode geometry.

In some examples, one or more of electrodes 232 and 234 may be suitable for sensing the ECAPs. For instance, electrodes 232 and 234 may sense the voltage amplitude of a portion of the ECAP signals, where the sensed voltage amplitude is a characteristic the ECAP signal. A sudden change in the sensed voltage amplitude of ECAP signals may be indicative of a movement of the trialing leads within patient 105. In some examples, processing circuitry of medical device 110 may measure a voltage across or a current through one or more of electrodes 232 and 234. A sudden change in the measured voltage across or current through the trialing leads or electrodes may be indicative of a movement of the trialing leads within patient 105.

Medical device 110 may include additional sensors within the housing of medical device 110 and/or coupled via one of leads 130 or other leads. In addition, medical device 110 may receive sensor signals wirelessly from remote sensors via telemetry circuitry 213, for example. In some examples, one or more of these remote sensors may be external to patient (e.g., carried on the external surface of the skin, attached to clothing, or otherwise positioned external to the patient). In some examples, signals from sensors 216 may indicate a position or body state (e.g., sleeping, awake, sitting, standing, or the like), and processing circuitry 214 may select target ECAP characteristic values according to the indicated position or body state.

Sensors 216 may include one or more sensing elements that sense values of a respective patient parameter. As described, electrodes 232 and 234 may be the electrodes that sense the parameter value of the ECAP elicited by informing control pulses. Sensor 216 may include one or more motion sensors, optical sensors, chemical sensors, temperature sensors, pressure sensors, strain sensors, humidity sensors, or any other types of sensors.

Although sensing circuitry 212 is incorporated into a common housing with stimulation generator 211 and processing circuitry 214 in FIG. 2, in other examples, sensing circuitry 212 may be in a separate housing from medical device 110 and may communicate with processing circuitry 214 via wired or wireless communication techniques. In some examples, sensing circuitry 212 may be a part of user device 160 or programmer 150, or any combination of medical device 110, user device 160, and/or programmer 150. Similarly, although sensors 216 are depicted within medical device 110 in FIG. 2, in some examples, sensors 216 may be integrated circuitry with user device 160 or programmer 150. In some examples, a plurality of sensors 216 are split between medical device 110, user device 160, and/or programmer 150.

Telemetry circuitry 213 supports wireless communication between medical device 110 and other computing devices, such as programmer 150, user device 160, or other computing devices under the control of processing circuitry 214. Processing circuitry 214 of medical device 110 may receive, as updates to programs, values for various stimulation parameters such as amplitude and electrode combination, from programmer 150 via telemetry circuitry 213. Updates to the therapy stimulation programs 217 and ECAP test stimulation programs 218 may be stored within memory 215. Telemetry circuitry 213 in medical device 110, as well as telemetry circuits in other devices and systems described herein, such as programmer 150, may accomplish communication by radiofrequency (RF) communication techniques. In addition, telemetry circuitry 213 may communicate with an external medical device programmer via proximal inductive interaction of medical device 110 with programmer 150. Programmer 150 may be one example of programmer 150 of FIG. 1. Accordingly, telemetry circuitry 213 may send information to programmer 150 and/or user device 160 on a continuous basis, at periodic intervals, or upon request from medical device 110 or the programmer.

Power source 219 delivers operating power to various components of medical device 110. Power source 219 may include a rechargeable or non-rechargeable battery and a power generation circuit to produce the operating power. Recharging may be accomplished through proximal inductive interaction between an external charger and an inductive charging coil within medical device 110. In other examples, traditional primary cell batteries may be used.

According to the techniques of the disclosure sensors 216 may comprise motion sensors including one or more accelerometers, strain gauges, microelectromechanical systems (MEMS) gyroscopes, thermometers, heart rate monitors, electrodes, or other sensors configured to measure a value for a sensed parameter of patient 105. Processing circuitry 214 may receive sensed parameter values from sensors 216, and determine whether the sensed parameter values are within a threshold range. Please note that the values chosen below are arbitrary and for illustration purposes only. For example, processing circuitry 214 may receive a vertical acceleration value of four-point-five m/s² from a vertical accelerometer of sensors 216. A first threshold range in memory for a vertical accelerometer may include a lower threshold value of negative four m/s² and an upper threshold value of four m/s². Processing circuitry 214 may determine that the sensed parameter value exceeds the upper threshold value, is outside the threshold range, and warn the patient that further activity could dislodge the trialing leads. The first threshold range may indicate the range outside which the trialing leads have a more significant chance of becoming dislodged. In some examples, a first threshold range may be defined such that values just outside of the range do not significantly increase the chances that leads may become dislodged. In these examples, when processing circuitry 214 determines that a sensed parameter value is outside the first threshold range, processing circuitry 214 may send a warning to the patient that increased activity levels may result in dislodged trialing leads before the patient actually engages in activities that result in a greater risk of dislodged trialing leads. A second threshold range may indicate the range outside of which the trialing leads have already become dislodged or are very likely to become dislodged. For example, data may show that acceleration higher than six m/s² dislodges the trialing leads in ninety percent of patients. Data may be collected from the medical devices of a plurality of patients with medical devices installed. In another example, a strain gauge may measure that the patient twisted their body such that the medical device 110 was further away from the insertion site of leads 130 than the length of leads 130 themselves, indicating the leads have been pulled free from their original positions.

In response to determining that the sensed parameter value is outside a first threshold range, processing circuitry 214 may communicate with user device 160 through telemetry circuitry 213. Processing circuitry may send an alert to user device 160, describing the measured parameter value, the first threshold range, and a warning that continued patient movement outside the threshold range may result in the trialing leads becoming dislodged. In some examples, the sensed parameter value is outside a first threshold range, and a second threshold range, where the second threshold range is larger than the first threshold range and contains the first threshold range. In response to determining that the sensed parameter value is outside the second threshold range, processing circuitry 214 may send an alert to user device 160, describing the measured parameter value, the threshold range, and a warning that the trialing leads have been, or have likely been dislodged. Given the possible overlap of thresholds (e.g., exceeding the second threshold range may inherently exceed the first threshold range), the alerts and/or warnings generated by processing circuitry 214 in response to determining that a sensed parameter value is outside a second threshold range may supersede alerts and/or warnings that would be generated in response to a sensed parameter value that is outside a first threshold range.

Although there are many references to two threshold ranges for a given sensed parameter value, in some examples there may be any number of threshold ranges for the parameter value. In some examples, processing circuitry may generate a different warning or alert for each threshold range of the number of threshold ranges for a given parameter value.

Although sensed parameter values are described throughout as being outside threshold ranges, in some examples, processing circuitry 214 may not assign a direction to parameter values sensed by structures of medical device 110. In these examples, a sensed parameter value may simply exceed a threshold value, where the threshold value is a discreet point, regardless of whether that threshold value represents a parameter value in a specific direction or not. In some examples, processing circuitry 214 may calculate a parameter value as a negative value, and discussion of a sensed parameter “exceeding” that value should be thought of in terms of magnitude. References to threshold “ranges” throughout contemplate the magnitude of threshold “values” being exceeded.

In some examples, processing circuitry 214 may identify an activity type based on sensed parameter values for patient 105. For example, as described above, processing circuitry may determine that different accelerometer readings correspond to jumping, dancing, or another activity type. Processing circuitry 214 may determine one or more motion threshold ranges for a sensed parameter value by retrieving the one or more motion threshold ranges from memory 215, where the one or more motion threshold ranges are associated with the activity type. Please note that the values chosen below are arbitrary and for illustration purposes only. For example, processing circuitry 214 may determine that the activity type is jumping, and retrieve, from a database in memory, motion threshold ranges corresponding to jumping acceleration, including a first motion threshold range, and a second motion threshold range. The motion threshold ranges corresponding to jumping may differ from motion threshold ranges corresponding to another activity type, like dancing. After retrieving the motion threshold ranges, processing circuitry 214 may determine whether a sensed parameter value is outside one or more of the motion threshold ranges.

When processing circuitry 214 determines that a sensed parameter value is outside a threshold range, processing circuitry 214 may save the value, as well as activity type, time stamp, and an alert, to a database in memory 215. Processing circuitry 214 may then output information from the database in memory 215 to a physician device, through telemetry circuitry 213, indicating that a motion occurred which could have dislodged the trialing leads. In some examples, processing circuitry may output an alert to the physician device that the trialing leads have likely been dislodged, or have been dislodged, and/or that therapy provided by medical device 110 may have been compromised.

In some examples, processing circuitry 214 is configured to measure an impedance signal within medical device 110. Processing circuitry may store impedance signals in a buffer and an algorithm executed on processing circuitry 214 of medical device 110 may continuously or periodically analyze the signals. A sudden change in the impedance of medical device 110 may be indicative of an activity level that has dislodged the trialing leads. If the impedance signal in medical device 110 is outside a threshold range, processing circuitry 214 may determine that trialing leads of medical device 110 have become dislodged. In response to determining that the trialing leads have become dislodged, processing circuitry 214 may send an alert to user device 160, describing the measured parameter value, the threshold range, and a warning that the trialing leads have been dislodged.

In some examples, processing circuitry is configured to measure an impedance of one or more electrodes in medical device 110. Movement of the electrodes on a neurostimulator closer to the target tissue may result in increased perception by the patient (e.g., possible painful sensations), and movement of the electrodes further from the target tissue may result in decreased efficacy of the therapy for the patient. As such, a large change in impedance signals for the one or more electrodes may be an indication that the trialing leads and electrodes have become dislodged.

Processing circuitry 214 may receive an electrode impedance threshold range comprising an upper electrode impedance threshold and a lower electrode impedance threshold from a database in memory 215, where the lower electrode impedance threshold is lower than the upper electrode impedance threshold. Processing circuitry may also measure an impedance signal of one or more electrodes in medical device 110 with one or more average impedance values, where the one or more average impedance values are below the upper electrode impedance threshold and above the lower electrode impedance threshold. As the buffer updates, processing circuitry 214 may update the one or more average impedance values. If the one or more average impedance values exceed the upper electrode impedance threshold, or fall below the lower electrode impedance threshold, processing circuitry may determine that the electrodes and trialing leads have become dislodged. In some examples, only the impedance value of one electrode exceeds an upper threshold or falls below a lower threshold, in which case processing circuitry 214 may determine that the single electrode is faulty, rather than that the trialing leads have been dislodged. In some examples, only the impedance values of the electrodes on one trialing lead exceed an upper threshold or fall below a lower threshold, in which case, processing circuitry 214 may determine that only the one trialing lead has become dislodged.

In some examples, processing circuitry 214 may use multiple sensed parameters to more accurately determine if the trialing leads have been dislodged. For example, if all electrodes of the one or more electrodes of medical device 110 exhibit an identical and simultaneous change in their respective impedance signals, and the identical change bring all of their respective impedance signals outside of a threshold range, processing circuitry 214 may have a high accuracy in its determination that the trialing leads have become dislodged. If fewer than all electrodes exhibit the identical, simultaneous change, or if the changes are not identical or simultaneous, a determination that the trialing leads have been dislodged may be less accurate. Processing circuitry 214 may record all instances of impedance values outside a threshold range in a database in memory 215, along with a time stamp, the impedance values for each electrode, and the threshold ranges.

While the above examples reference impedance, processing circuitry 214 may instead, or in addition, be configured to measure a different parameter (e.g., a resistance of, voltage across, or current through one or more electrodes in medical device 110). A large change in resistance signals for the one or more electrodes may be an indication that the trialing leads and electrodes have become dislodged. Processing circuitry 214 may determine if resistance values are outside resistance threshold ranges and generate information indicating a status of the trialing leads based on determining that the resistance values are outside resistance threshold ranges.

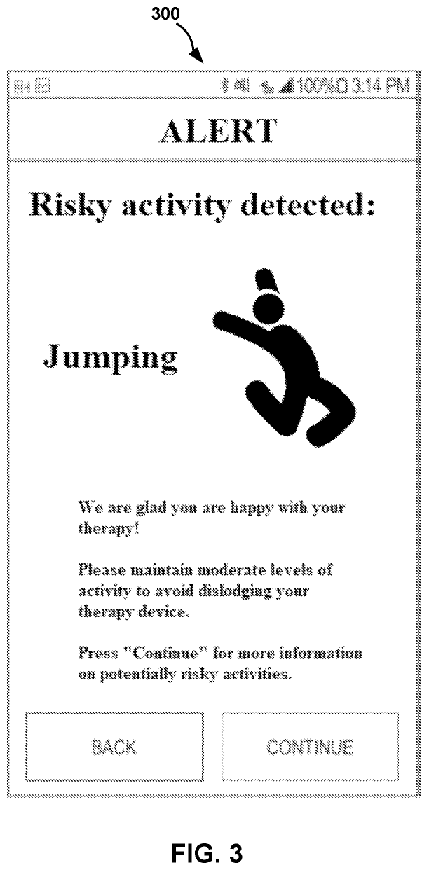

FIG. 3 is a conceptual diagram illustrating an example alert on a user interface 300 to warn a user of excessive activity levels.

In response to determining that a sensed parameter value is outside a threshold range, processing circuitry 214 may output an alert warning patient 105 that patient movement could dislodge the trialing leads. The alert may appear on a user device 160 of patient 105. In some examples processing circuitry 214 may identify an activity type (e.g., jumping, dancing, running), and output the identified activity type with the alert.

The alert may indicate that it is an alert through words, sounds, or any other method available on user device 160 for capturing patient 105's attention. For example, the alert may appear as a notification on a user device 160 of patient 105, with all capital letters indicating that it is an “alert,” “warning,” or some other indication of urgency. In some examples, the alert includes a sound, tone, or alarm, that audibly alerts patient 105 of the alert. In some examples the alert includes a vibration of user device 160. In some examples, the vibration, sound, and/or text alert may persist (or repeat at predetermined intervals) until the sensed parameter value is within the threshold range.

In some examples, the alert includes the sensed parameter value, threshold range, activity type, and warning that continued activity may dislodge the trialing leads. In some examples, the alert includes only one or more of the previously mentioned items in any combination.

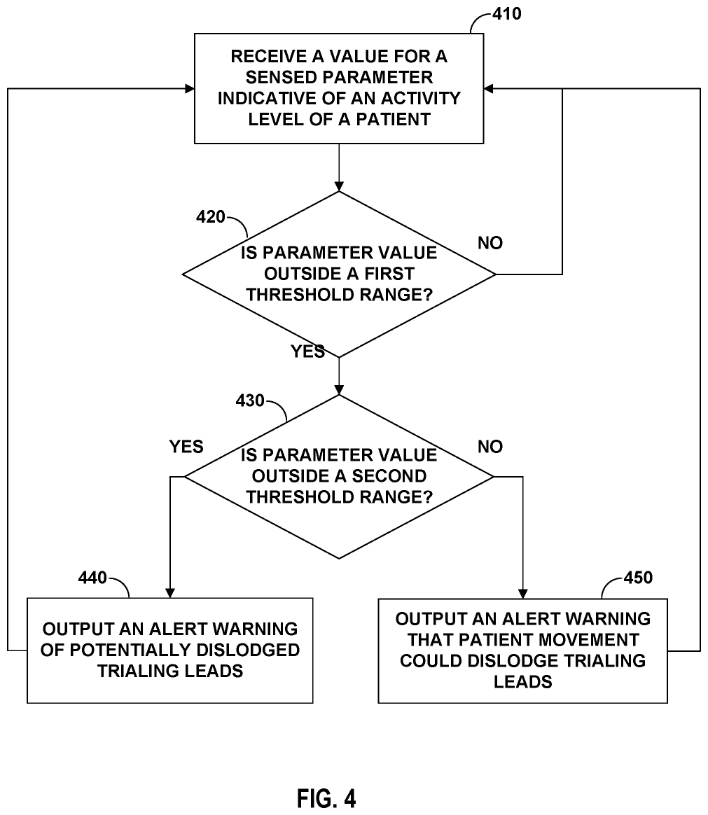

FIG. 4 is a flowchart illustrating an example technique for preventing dislodged medical devices according to the techniques of this disclosure.

An algorithm executed on processing circuitry 214 of medical device 110 may receive a value for a sensed parameter indicative of an activity level of patient 105 (410). The sensed parameter may be sensed by one or more sensors 216 which may include one or more motion sensors, optical sensors, chemical sensors, temperature sensors, pressure sensors, strain sensors, or any other types of sensors configured to sense a parameter indicative of an activity level of patient 105. In some examples, the sensed parameter may be an impedance, resistance, or ECAP signal measured on the leads 130 of medical device 110. Based on the sensors 216 and sensed parameters, processing circuitry 214 may determine an activity type for the sensed parameters. For example, processing circuitry 214 may receive a vertical acceleration signal from a vertical accelerometer, a frontal acceleration signal from a frontal accelerometer, a temperature signal from a thermometer, and a heart rate signal from a heart rate monitor. The vertical acceleration signal may indicate a small, regular oscillation in vertical acceleration, the frontal acceleration signal may indicate regular, gradual changes in frontal acceleration, the temperature signal may indicate a heightened body temperature, and the heart rate monitor may indicate an increased heart rate. Based on these signals, processing circuitry may determine that patient 105 is jogging.

Processing circuitry 214 may receive one or more threshold ranges for one or more sensed parameters from a database in memory 215, where the one or more threshold ranges may be associated with the activity type. For example, processing circuitry 214 may receive a first vertical acceleration threshold range, a second vertical acceleration threshold range, a temperature threshold range, and a heart rate threshold range.

Processing circuitry 214 may determine if one or more sensed parameters is outside the one or more corresponding threshold ranges (420). For example, processing circuitry may receive a vertical acceleration value from sensors 216. Processing circuitry may determine that the vertical acceleration value exceeds the upper value of the first and/or second vertical acceleration threshold ranges, and output an alert to the patient.

In some examples, processing circuitry 214 may require that multiple sensed parameters are outside their corresponding threshold ranges in order to output an alert. For example, processing circuitry 214 may receive a vertical acceleration value that is outside the first vertical acceleration threshold range, but not outside the second vertical acceleration threshold range. Processing circuitry may also receive a temperature value that is within a temperature threshold range, and a heart rate value that is within a heart rate threshold range. While the acceleration value has exceeded an acceleration threshold, the temperature and heart rate values are within normal ranges, indicating that the acceleration value may be the result of something other than patient 105's own movement (e.g., patient 105 is sitting in a plane that is taking off, or a vehicle accelerating up a hill).

Processing circuitry 214 may determine if a sensed parameter that is outside a first threshold range is also outside a second threshold range (430). For example, processing circuitry 214 may receive a vertical acceleration value that is higher than the upper threshold of both the first and second vertical acceleration ranges, and may determine that the vertical acceleration value is outside both the first and second vertical acceleration threshold ranges. In some examples, a value that exceeds the upper threshold of the second threshold range may inherently exceed the upper threshold of the first threshold range. The alerts and/or warnings generated by processing circuitry 214 in response to determining that a sensed parameter value is outside a second threshold range may supersede alerts and/or warnings that would be generated in response to a sensed parameter value that is outside a first threshold range.

If processing circuitry 214 determines that a sensed parameter value is outside a first threshold range, but not a second threshold range, processing circuitry 214 may generate information indicating a status of one or more trialing leads by outputting an alert warning that further patient movement could dislodge trailing leads (450). In some examples, the first threshold range represents a range outside of which the trialing leads have a more significant chance of becoming dislodged. In some examples, a first threshold range may be defined such that values just outside of the range do not significantly increase the chances that leads may become dislodged. In these examples, when processing circuitry 214 determines that a sensed parameter value is outside the first threshold range, processing circuitry 214 may send a warning to the patient that increased activity levels may result in dislodged trialing leads before the patient engages in activities that result in a greater risk of dislodged trialing leads, or engages in activities that result in dislodged trialing leads.

If processing circuitry 214 determines that a sensed parameter value is outside a first threshold range and a second threshold range, processing circuitry 214 may generate information indicating a status of one or more trialing leads by outputting an alert warning that patient movement has potentially dislodged the trailing leads of medical device 110 (440). The second threshold range may indicate the range outside of which the trialing leads have already become dislodged or are very likely to become dislodged. In some examples, two different sensed parameter values may be outside two different threshold ranges that, together, indicate the trialing leads have been dislodged. For example, processing circuitry 214 may receive a first vertical acceleration threshold range, a second vertical acceleration threshold range, a vertical acceleration value outside the first vertical acceleration range but not the second vertical acceleration range, an impedance threshold range, and an impedance value outside of the impedance threshold range. Although the vertical acceleration value is not outside the second vertical acceleration threshold range, because the impedance value is outside the impedance threshold range, processing circuitry 214 may determine that the trialing leads have likely been dislodged and output an alert to patient 105 that the trialing leads are likely dislodged.

In some examples, processing circuitry may measure a sensed parameter of one or more electrodes in medical device 110. In some examples, the sensed parameter is an impedance. Processing circuitry 214 may receive an electrode impedance threshold range comprising an upper electrode impedance threshold and a lower electrode impedance threshold from a database in memory 215, where the lower electrode impedance threshold is lower than the upper electrode impedance threshold. Processing circuitry may also measure one or more impedance signals of one or more electrodes in medical device 110. If the one or more impedance signals exceed the upper electrode impedance threshold, or fall below the lower electrode impedance threshold, processing circuitry may determine that the electrodes and trialing leads have become dislodged. In some examples, only the impedance value of one electrode exceeds an upper threshold or falls below a lower threshold, in which case processing circuitry 214 may determine that the single electrode is faulty, rather than that the trialing leads have been dislodged. In some examples, only the impedance values of the electrodes on one trialing lead exceed an upper threshold or fall below a lower threshold, in which case, processing circuitry 214 may determine that only the one trialing lead has become dislodged.

While the above examples reference impedance, processing circuitry 214 may instead, or in addition, measure other parameters of the one or more electrodes in medical device 110 (e.g., resistance). A large change in resistance signals for the one or more electrodes may be an indication that the trialing leads and electrodes have become dislodged. Processing circuitry 214 may determine if resistance values are outside resistance threshold ranges and generate information indicating a status of the trialing leads based on determining that the resistance values are outside resistance threshold ranges.

Although this disclosure sometimes refers to a sensed parameter value “exceeding” an upper threshold range value, in some examples, a sensed parameter value that is lower than a lower threshold range value may trigger processing circuitry 214 to output an alert warning that patient movement could, or potentially has, dislodged trialing leads. For example, processing circuitry 214 may receive an ECAP amplitude value below a lower threshold of an ECAP threshold range. Processing circuitry 214 may determine that the ECAP amplitude value below the lower ECAP threshold value is more likely indicative of noise than an actual ECAP reading.

In another example, processing circuitry 214 may generate information indicating that there is a low confidence in an accuracy of a signal sensed by the one or more trialing leads. For example, as described above, an ECAP amplitude below the lower ECAP threshold value is more likely indicative of noise than an actual ECAP reading, thus processing circuitry 214 may output an alert that there is low confidence in the accuracy of the ECAP signal, as well as an alert that the trialing leads have likely been dislodged. Processing circuitry 214 may output the alert to one or more of a user device 160, programmer 150, or other device by way of telemetry circuitry 213 to warn a patient and/or physician of the potentially inaccurate signals and the potentially dislodged trialing leads.

Processing circuitry 214 may continue to monitor sensed parameter values after outputting an alert, and may continue to output an alert until the sensed parameter values are within the proper limits, or the alert has been silenced by a user of user device 160.

When processing circuitry 214 determines that a sensed parameter value is outside a threshold range, processing circuitry 214 may save the value, as well as activity type, time stamp, and an alert, to a database in memory 215. Processing circuitry 214 may output information from the database in memory 215 to a physician device, through telemetry circuitry 213, indicating that a motion occurred which could have dislodged the trialing leads. In some examples, processing circuitry may output an alert to the physician device that the trialing leads have likely been dislodged, and/or that therapy provided by medical device 110 may have been compromised.

The following examples are described herein.

Example 1: A medical system includes: one or more trialing leads implanted within a patient; one or more sensors configured to determine a value for a sensed parameter indicative of an activity level of the patient; and processing circuitry. The processing circuitry may be configured to: receive the value from the one or more sensors; determine whether the value is outside a threshold range; and in response to determining that the value is outside the threshold range, generate information indicating a status of the one or more trialing leads.

Example 2: The medical system of example 1, wherein to generate information indicating the status of the one or more trialing leads, the processing circuitry is configured to output an alert warning that patient movement could dislodge the one or more trialing leads.

Example 3: The medical system of any of examples 1 or 2, wherein the threshold range comprises a first threshold range and the alert comprises a first alert, and the processing circuitry is further configured to: determine whether the value is outside a second threshold range; and in response to determining that the value is outside the second threshold range, output a second alert warning of potentially dislodged trialing leads.

Example 4: The medical system of any of examples 1-3, wherein the sensors comprise one or more strain gauges.

Example 5: The medical system of any of examples 1-3, wherein the sensors comprise one or more accelerometers.

Example 6: The medical system of any of examples 1-5, wherein to generate information indicating the status of the one or more trialing leads, the processing circuitry is configured to generate information indicating that there is low confidence in an accuracy of a signal sensed by the one or more trialing leads.

Example 7: The medical system of any of examples 1-6, further comprising a memory, and wherein to determine whether the value is outside the threshold range, processing circuitry is further configured to: identify an activity type; retrieve one or more motion threshold ranges from the memory, wherein the motion threshold ranges retrieved are associated with the activity type; and determine whether the value is outside one or more of the motion threshold ranges associated with the activity type.

Example 8: The medical system of example 7, wherein to generate information indicating the status of the one or more trialing leads, the processing circuitry is configured to: save the value, activity type, and alert to a database in the memory with a time stamp; and output information from the database in memory, for a physician, warning of potentially dislodged trialing leads.

Example 9: The medical system of any of examples 1-8, further comprising a neurostimulator, and wherein the processing circuitry is further configured to: determine an impedance signal in the neurostimulator; determine whether the impedance signal is outside the threshold range; responsive to determining that the impedance signal is outside the threshold range, generate information indicating likelihood that the one or more trialing leads dislodged.

Example 10: The medical system of example 9, further comprising a plurality of stimulation electrodes on the one or more trialing leads, and wherein the processing circuitry is further configured to: determine the impedance on each of the plurality of stimulation electrodes; and determine whether the impedance signal is outside the threshold range for each of the plurality of stimulation electrodes; and responsive to determining that the impedance signal is outside the threshold range for a predetermined number of the plurality of stimulation electrodes, generate information indicating a likelihood that the one or more trialing leads dislodged.

Example 11: The medical system of any of examples 1-10, further comprising a medical device coupled to the one or more trialing leads, wherein the medical device includes the one or more sensors and the processing circuitry.

Example 12: The medical system of any of examples 1-10, further comprising a wearable device and a medical device coupled to the one or more trialing leads, wherein at least one of: the wearable device includes both the one or more sensors and the processing circuitry; the medical device includes both the one or more sensors and the processing circuitry; or the medical device includes one of the one or more sensors or the processing circuitry, and the wearable device includes the other one of the one or more sensors or the processing circuitry.

Example 13: A method comprising: receiving from one or more sensors, by processing circuitry, a value for a sensed parameter indicative of an activity level of a patient; determining, by processing circuitry, that the value is outside a threshold range; and responsive to determining that the value is outside the threshold range, generating, by processing circuitry, information indicating a status of one or more trialing leads implanted within the patient.

Example 14: The method of example 13, wherein generating information indicating a status of the one or more trialing leads comprises outputting an alert warning that patient movement could dislodge the one or more trialing leads.

Example 15: The method of any of examples 13 or 14, wherein the threshold range comprises a first threshold range and the alert comprises a first alert, further comprising: determining, by processing circuitry, that the value is outside a second threshold range; and responsive to determining that the value is outside the second threshold range, outputting, by processing circuitry, a second alert warning of potentially dislodged trialing leads.

Example 16: The method of any of examples 13-15, wherein generating information indicating a status of one or more trialing leads comprises generating information indicating that there is a low confidence in an accuracy of a signal sensed by the one or more trialing leads.

Example 17: The method of any of examples 13-16, wherein determining that the value is outside the threshold range comprises: identifying, by processing circuitry, an activity type; retrieving, by processing circuitry, one or more motion threshold ranges from a memory, wherein the motion threshold ranges retrieved are associated with the activity type; and determining, by processing circuitry, that the value is outside one or more of the motion threshold ranges associated with the activity type.

Example 18: The method of example 17, further comprising: saving, by processing circuitry, the value, activity type, and alert to a database in the memory with a time stamp; and outputting information from the database in memory, by processing circuitry, for a physician, warning of potentially dislodged trialing leads.

Example 19: The method of any of examples 13-18, further comprising: determining, by processing circuitry, an impedance signal in a neurostimulator; determining, by processing circuitry, that the impedance signal is outside the threshold range; responsive to determining that the impedance signal is outside the threshold range, generating, by processing circuitry, information indicating a likelihood that the one or more trialing leads dislodged.

Example 20: The method of example 19 further comprising: determining, by processing circuitry, the impedance on each of a plurality of stimulation electrodes; and determining, by processing circuitry, that the impedance signal is outside the threshold range for a predetermined number of the plurality of stimulation electrodes; and responsive to determining that the impedance signal is outside the threshold range for the predetermined number of the plurality of stimulation electrodes, generating, by processing circuitry, information indicating a likelihood that the one or more trialing leads dislodged.

Example 21. A computer-readable storage medium storing instructions thereon that when executed cause one or more processors to: receive from one or more sensors a value for a sensed parameter indicative of an activity level of a patient; determine that the value is outside a threshold range; and responsive to determining that the value is outside the threshold range, generate information indicating a status of one or more trialing leads implanted within the patient.

Example 22. The computer-readable storage medium of example 21, further comprising instructions that cause the one or more processors to perform the method of any of examples 13-20.

Example 23. A device comprising means for receiving from one or more sensors a value for a sensed parameter indicative of an activity level of a patient; means for determining that the value is outside a threshold range; and means for generating information indicating a status of one or more trialing leads implanted within the patient responsive to determining that the value is outside the threshold range.

Example 24. The device of example 23, further comprising means for performing the method of any of examples 13-20.

In one or more examples, the functions described may be implemented in hardware, software, firmware, or any combination thereof. If implemented in software, the functions may be stored on or transmitted over a computer-readable medium as one or more instructions or code, and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can include RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more DSPs, general purpose microprocessors, ASICs, FPGAs, or other equivalent integrated or discrete logic circuitry, as well as any combination of such components. Accordingly, the term “processor,” as used herein may refer to any of the foregoing structures or any other structure suitable for implementation of the techniques described herein. In addition, in some aspects, the functionality described herein may be provided within dedicated hardware and/or software modules. Also, the techniques could be fully implemented in one or more circuits or logic elements.

The techniques of this disclosure may be implemented in a wide variety of devices or apparatuses, including a wireless communication device or wireless handset, a microprocessor, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

Various examples have been described. These and other examples are within the scope of the following claims.

What is claimed is:

1. A medical system comprising:
one or more trialing leads implanted within a patient;
one or more sensors configured to determine a value for a sensed parameter indicative of an activity level of the patient; and
processing circuitry configured to:
receive the value from the one or more sensors;
determine whether the value is outside a threshold range; and
in response to determining that the value is outside the threshold range, generate information indicating a status of the one or more trialing leads.

2. The medical system of claim 1, wherein to generate information indicating the status of the one or more trialing leads, the processing circuitry is configured to output an alert warning that patient movement could dislodge the one or more trialing leads.

3. The medical system of claim 1, wherein the threshold range comprises a first threshold range and the alert comprises a first alert, and the processing circuitry is further configured to:
determine whether the value is outside a second threshold range; and
in response to determining that the value is outside the second threshold range, output a second alert warning of potentially dislodged trialing leads.

4. The medical system of claim 1, wherein the sensors comprise one or more strain gauges or one or more accelerometers.

5. The medical system of claim 1, wherein to generate information indicating the status of the one or more trialing leads, the processing circuitry is configured to generate information indicating that there is low confidence in an accuracy of a signal sensed by the one or more trialing leads.

6. The medical system of claim 1, further comprising a memory, and wherein to determine whether the value is outside the threshold range, processing circuitry is further configured to:
identify an activity type;
retrieve one or more motion threshold ranges from the memory, wherein the motion threshold ranges retrieved are associated with the activity type; and
determine whether the value is outside one or more of the motion threshold ranges associated with the activity type.

7. The medical system of claim 6, wherein to generate information indicating the status of the one or more trialing leads, the processing circuitry is configured to:
save the value, activity type, and alert to a database in the memory with a time stamp; and
output information from the database in memory, for a physician, warning of potentially dislodged trialing leads.

8. The medical system of claim 1, further comprising a neurostimulator, and wherein the processing circuitry is further configured to:
determine an impedance signal in the neurostimulator;
determine whether the impedance signal is outside the threshold range;
responsive to determining that the impedance signal is outside the threshold range, generate information indicating likelihood that the one or more trialing leads dislodged.

9. The medical system of claim 8, further comprising a plurality of stimulation electrodes on the one or more trialing leads, and wherein the processing circuitry is further configured to:
determine the impedance on each of the plurality of stimulation electrodes; and
determine whether the impedance signal is outside the threshold range for each of the plurality of stimulation electrodes; and
responsive to determining that the impedance signal is outside the threshold range for a predetermined number of the plurality of stimulation electrodes, generate information indicating a likelihood that the one or more trialing leads dislodged.

10. The medical system of claim 1, further comprising a medical device coupled to the one or more trialing leads, wherein the medical device includes the one or more sensors and the processing circuitry.

11. The medical system of claim 1, further comprising a wearable device and a medical device coupled to the one or more trialing leads, wherein at least one of:
the wearable device includes both the one or more sensors and the processing circuitry;
the medical device includes both the one or more sensors and the processing circuitry; or
the medical device includes one of the one or more sensors or the processing circuitry, and the wearable device includes the other one of the one or more sensors or the processing circuitry.

12. A method comprising:
receiving from one or more sensors, by processing circuitry, a value for a sensed parameter indicative of an activity level of a patient;
determining, by processing circuitry, that the value is outside a threshold range; and
responsive to determining that the value is outside the threshold range, generating, by processing circuitry, information indicating a status of one or more trialing leads implanted within the patient.

13. The method of claim 12, wherein generating information indicating a status of the one or more trialing leads comprises outputting an alert warning that patient movement could dislodge the one or more trialing leads.

14. The method of claim 12, wherein the threshold range comprises a first threshold range and the alert comprises a first alert, further comprising:
determining, by processing circuitry, that the value is outside a second threshold range; and
responsive to determining that the value is outside the second threshold range, outputting, by processing circuitry, a second alert warning of potentially dislodged trialing leads.

15. The method of claim 12, wherein generating information indicating a status of one or more trialing leads comprises generating information indicating that there is a low confidence in an accuracy of a signal sensed by the one or more trialing leads.

16. The method of claim 12, wherein determining that the value is outside the threshold range comprises:
identifying, by processing circuitry, an activity type;
retrieving, by processing circuitry, one or more motion threshold ranges from a memory, wherein the motion threshold ranges retrieved are associated with the activity type; and
determining, by processing circuitry, that the value is outside one or more of the motion threshold ranges associated with the activity type.

17. The method of claim 16, further comprising:
saving, by processing circuitry, the value, activity type, and alert to a database in the memory with a time stamp; and
outputting information from the database in memory, by processing circuitry, for a physician, warning of potentially dislodged trialing leads.

18. The method of claim 12, further comprising:
determining, by processing circuitry, an impedance signal in a neurostimulator;
determining, by processing circuitry, that the impedance signal is outside the threshold range;
responsive to determining that the impedance signal is outside the threshold range, generating, by processing circuitry, information indicating a likelihood that the one or more trialing leads dislodged.

19. The method of claim 18 further comprising:
determining, by processing circuitry, the impedance on each of a plurality of stimulation electrodes; and
determining, by processing circuitry, that the impedance signal is outside the threshold range for a predetermined number of the plurality of stimulation electrodes; and
responsive to determining that the impedance signal is outside the threshold range for the predetermined number of the plurality of stimulation electrodes, generating, by processing circuitry, information indicating a likelihood that the one or more trialing leads dislodged.

20. A computer-readable storage medium storing instructions thereon that when executed cause one or more processors to:
receive from one or more sensors a value for a sensed parameter indicative of an activity level of a patient;
determine that the value is outside a threshold range; and
responsive to determining that the value is outside the threshold range, generate information indicating a status of one or more trialing leads implanted within the patient.
